(19) United States
(12) Patent Application Publication
(10) Pub. No.: US 2025/0312571 A1
(43) Pub. Date: Oct. 9, 2025

(54) GUIDING CATHETER

(71) Applicant: BIOTRONIK AG, Buelach (CH)

(72) Inventor: Dragana Simic, Niederhasli (CH)

(21) Appl. No.: 18/869,505

(22) PCT Filed: Jun. 9, 2023
(86) PCT No.: PCT/EP2023/065470

§ 371 Date: Nov. 26, 2024

(30) Foreign Application Priority Data
Jun. 15, 2022 (EP) 22179233.6

(51) Int. Cl. A61M 25/06; A61M 25/00; A61M 25/01; A61M 25/09

(52) U.S. Cl. CPC A61M 25/0662; A61M 2025/0006; A61M 2025/0175; A61M 2025/0188; A61M 2025/0681; A61M 2025/09166

(57) ABSTRACT

A guiding catheter includes an outer shaft defining a first guiding lumen in an interior of the outer shaft. An inner shaft is movably arranged at least partially in the first guiding lumen. The inner shaft defines a second guiding lumen in an interior of the inner shaft. A dividing device is arranged at a proximal end region of the guiding catheter. The dividing device includes a first port, and a second port angularly arranged with respect to each other. Both the first port and the second port provide access to the first guiding lumen. The inner shaft extends through the second port into the first guiding lumen. The inner shaft includes at least a section-wise slit along a longitudinal extension direction (F) of the inner shaft. The slit provides access to the second guiding lumen and enables a working catheter to enter the second guiding lumen from the first guiding lumen. The inner shaft has a length exceeding a length of the outer shaft.

### PRIORITY CLAIM

This application is a 35 U.S.C. 371 US National Phase and claims priority under 35 U.S.C. § 119, 35 U.S.C. 365(b) and all applicable statutes and treaties from prior PCT Application PCT/EP2023/065470, which was filed Jun. 9, 2023, which application claimed priority from EP application Ser. No. 22179233.6, which was filed Jun. 15, 2022.

### FIELD OF THE INVENTION

The present invention relates to a guiding catheter used to guide a working catheter.

### BACKGROUND

A guiding catheter is used to guide a working catheter to a site of operation or implantation. If a working catheter is to be guided to a remotely located site of operation, such as the coronary or peripheral vasculature, a guiding catheter needs to be used in combination with a guide extension to bridge the long distance between an entry opening through which the guiding catheter is guided into the body of the patient and the coronary or peripheral vasculature at which a manipulation with the working catheter is to be performed. Thus, according to prior art techniques, two catheters are necessary to reach specific vascular regions. The handling of two separate catheters makes the manipulation procedure much more complex than in case of using only a single catheter. Furthermore, upon inserting or retracting a balloon or a stent with the working catheter, further complications may occur. To give an example, a stent or balloon may be damaged or even destroyed when being guided without specific care over a connection site between the guiding catheter and the guide extension.

Considering that an operator needs not only to handle the guiding catheter and the guide extension, but also two guide wires, the complexity of the whole manipulation procedure becomes even more apparent. Finally, the manufacturing costs for two separate catheters including their packaging results in high manufacturing costs. If the diameter of the guiding catheter and the guide extension are not well adjusted to each other, both instruments may be damaged prior to accomplish the task of guiding a working catheter in their interior.

### SUMMARY OF THE INVENTION

A guiding catheter of the invention enables an easier guiding of a working catheter even to remote locations and that reduces the risk of damages of the working catheter. A preferred guiding catheter includes an outer shaft defining a first guiding lumen in an interior of the outer shaft. An inner shaft is movably arranged at least partially in the first guiding lumen. The inner shaft defines a second guiding lumen in an interior of the inner shaft. A dividing device is arranged at a proximal end region of the guiding catheter. The dividing device includes a first port, and a second port angularly arranged with respect to each other. Both the first port and the second port provide access to the first guiding lumen. The inner shaft extends through the second port into the first guiding lumen. The inner shaft includes at least a section-wise slit along a longitudinal extension direction (F) of the inner shaft. The slit provides access to the second guiding lumen and enables a working catheter to enter the second guiding lumen from the first guiding lumen. The inner shaft has a length exceeding a length of the outer shaft.

### BRIEF DESCRIPTION OF THE DRAWINGS

Further details of aspects of the present invention will be explained in the following making reference to exemplary embodiments and accompanying Figures. In the Figures:

FIG. 1A shows an embodiment of a guiding catheter in a first operational state;

FIG. 1B shows the guiding catheter of FIG. 1A in a second operational state;

FIG. 2 shows a first detail of the guiding catheter of FIG. 1A; and

FIG. 3 shows a second detail of the guiding catheter of FIG. 1A.

### DETAILED DESCRIPTION OF THE PREFERRED EMBODIMENTS

A guiding catheter of the invention includes an outer shaft that defines a first guiding lumen in an interior of the outer shaft. The guiding catheter further includes an inner shaft. The inner shaft is at least section-wise arranged within the first guiding lumen in a movable manner. Typically, it can be moved by a translational and optionally also by a rotational movement with respect to the outer shaft. The inner shaft defines a second guiding lumen in an interior of the inner shaft.

The guiding catheter further includes a dividing device arranged in a proximal end region of the guiding catheter. The dividing device has a first port and a second port that are angularly arranged to each other. In this context, both the first port and the second port provide access to the first guiding lumen. Thus, if an object is guided through the first port or through the second port, it will reach the first guiding lumen.

According to an aspect of the presently claimed invention, the inner shaft extends through the second port into the first guiding lumen. In this context, the inner shaft includes at least partially a slit along a longitudinal extension direction of the inner shaft. This slit fully penetrates a wall of the inner shaft so that it enables an access from an outside of the inner shaft to the second guiding lumen (being inside the inner shaft). Therefore, the slit enables a working catheter to enter the second guiding lumen from the first guiding lumen, i.e., from an outside of the inner shaft.

The presently claimed guiding catheter can replace prior art guiding catheters and prior art guide extensions at the same time. By advancing the inner shaft relatively to the outer shaft in a distal direction, an extension of the effective length of the guiding catheter can be reached. In doing so, only a single instrument, namely the guiding catheter itself, needs to be manipulated. If the inner shaft is advanced over a distal terminus of the outer shaft, the effective length of the guiding catheter is increased. Then, the guiding catheter can guide a working catheter over a longer distance to the desired site of action. If no such extension of the effective length of the guiding catheter is needed, it can also be used in its standard length. In this case, the inner shaft is arranged inside the first guiding lumen such that it does not protrude from the distal terminus of the outer shaft. Expressed in other words, the guiding catheter includes a built-in extension mechanism.

It is immediately apparent that the presently claimed and described guiding catheter can be handled much easier than prior art systems employing a guiding catheter and a separate guide extension.

In an embodiment, the slit is present in the proximal end region of the inner shaft. In this context, the slit has a length lying in a range of at least 5 cm, e.g. from 5 cm to 50 cm, in particular of from 10 cm to 45 cm, in particular of from 15 cm to 40 cm, in particular of from 20 cm to 35 cm or in particular of from 25 cm to 30 cm. The length of the slit determines the possible extension of the effective length of the guiding catheter, since the inner shaft can be moved with respect to the outer shaft about a length that corresponds to the length of the slit, while maintaining full operational function of the guiding catheter. A length of the slit lying in a range of from 15 cm to cm (i.e., around 20 cm) is particularly appropriate and can cover most applications of the guiding catheter.

In an embodiment, the slit is present in the proximal end region of the inner shaft starting from and including a proximal terminus of the inner shaft. To give an example, the slit may be positioned in a region over 15 to 25 cm starting from the proximal terminus of the inner shaft. It will then be possible to separate a first wall section lying on a first side of the slit and a second wall section lying on a second side of the slit to access the second guiding lumen. Such a separation of the two wall sections will be possible in the whole proximal end region in which the slit is arranged, i.e., including the proximal terminus of the inner shaft.

In an embodiment, the inner shaft includes or essentially consists of a polyether block amide (PEBA). Such a thermoplastic elastomer, which is also known under the trademark Pebax, has a high flexibility so that it can be easily guided within the first guiding lumen. At the same time, it has a sufficiently high stability to be able to safely guide a working catheter within the second guiding lumen.

In an embodiment, the dividing device includes an access facilitation device. This access facilitation device enables a facilitated access to the second guiding lumen through the slit. For this purpose, the access facilitation device typically gets into direct contact with the inner shaft and enables the avoidance of a direct contact between a catheter guided within the second guiding lumen and a wall of the inner shaft in the region of the slit. Thus, the access facilitation device is able to shield a working catheter guided within the second guiding lumen from undesired mechanical impacts by the wall of the inner shaft.

In an embodiment, the access facilitation device includes a tip that penetrates the slit and increases an area of an opening in a wall of the inner shaft formed by the slit. In this embodiment, the access facilitation device serves for strutting apart the wall of the inner shaft in the region of the slit and thus facilitates an access of the catheter through the opening formed by the slit into the second guiding lumen. In an embodiment, the access facilitation device is a hollow body. In other embodiments, the access facilitation device has a shield-like shape without forming a hollow body.

In an embodiment, the access facilitation device is at least partially arranged inside the first port. By such an arrangement, the access facilitation device particularly facilitates the access into the second guiding lumen by the working catheter introduced into the guiding catheter through the first port. The access facilitation device may be designed as sleeve arranged inside the first port.

In an embodiment, the second port includes a fixing device for fixing the inner shaft relatively to the dividing device and the outer shaft. Such a fixing device makes it possible to allow, restrict or prohibit a relative movement between the inner shaft and the outer shaft. To give an example, the fixing device can be designed as twist lock. The fixing device is configured to have an open position, in which a relative movement between the inner shaft and the outer shaft is possible (so that the effective guiding length of the guiding catheter can be adapted to the present needs) and a closed position, in which a relative movement between the inner shaft and the outer shaft is not possible. In case that only some further slight movements of the inner shaft are necessary, the twist lock can be somewhat tightened to restrict the relative movement between the inner shaft and the outer shaft, while not fully prohibiting such a movement. Once having positioned the inner shaft such that no further movement of the inner shaft with respect to the outer shaft is necessary or desired, the twist lock may be fully fastened so that a relative movement between the inner shaft and the outer shaft is prohibited. Then, the effective guiding length of the guiding catheter remains stable and the operator of the guiding catheter does no longer need his or her hands for keeping the inner shaft in place. This significantly facilitates any manipulations with a working catheter guided through the guiding catheter to the intended site of action.

In an embodiment, the guiding catheter includes an auxiliary wire guided within the second lumen. This auxiliary wire serves for stabilizing the inner shaft and allows an easier relative movement between the inner shaft and the outer shaft. To be more precise, the auxiliary wire stabilizes the inner shaft particularly in that region in which the inner shaft protrudes over the distal end of the outer shaft and is thus not supported by the outer shaft. The auxiliary wire may be designed like a standard guide wire known from prior art. The auxiliary wire can be made from the same materials as a guide wire known from prior art. It should be noted that even in case that an auxiliary wire is used, no wire will be guided in an intermediate space between an outside of the inner shaft and an inside of the outer shaft. Rather, the auxiliary wire is only guided within the second guiding lumen so that it does not impart a relative movement between the inner shaft and the outer shaft.

In an embodiment, the inner shaft has a length that exceeds a length of the outer shaft, e.g. by at least 10%, in particular by a value lying in a range of from 10% to 50%, in particular of from 15% to 45%, in particular of from 20% to 40%, in particular of from 25% to 35% or in particular of from 30% to 35%. To give some specific examples, the outer shaft has, in an embodiment, a length lying in a range of from 90 cm to 120 cm, in particular of from 100 cm to 110 cm. The length of the inner shaft exceeding the length of the outer shaft enables an extension of the guiding catheter. Thus, the guiding catheter can be used as a guide extension catheter.

In an embodiment, the inner shaft has the same diameter over its entire length. Then, any steps within the inner shaft are avoided. This facilitates an advancement and/or retraction of a working catheter including a stent or a balloon within the second guiding lumen. By providing a fully assembled guiding catheter with an inner shaft having a diameter being adapted to a diameter of the outer shaft, inappropriate choices by an operator with respect to the diameters of an inner and an outer shaft are avoided. According to prior art techniques, sometimes inappropriate guiding extensions are paired with a guiding catheter, leading to severe damages of a working catheter guided through the guiding catheter and the guiding extension.

In an embodiment, the outer diameter of the inner shaft corresponds to a value lying in a range of from 1 mm to 5 mm, in particular of from 1.5 mm to 4.5 mm, in particular of from 2 mm to 4 mm, in particular of from 2.5 mm to 3.5 mm, e.g., to 5 F (1.67 mm) or 6 F (2 mm). In this embodiment, a diameter of the outer shaft is bigger than the outer diameter of the inner shaft and corresponds to a value lying in a range of from 1.5 mm to 5.5 mm, in particular of from 2 mm to 5 mm, in particular of from 2.5 mm to 4.5 mm, in particular of from 3 mm to 4 mm, e.g., 6 F (2 mm) or 7 F (2.33 mm), respectively. Such a slight difference in size allows an easy relative movement of the inner shaft with respect to the outer shaft, but yet still allows the support of the inner shaft by the outer shaft.

In an embodiment, an angle between the first port and the second port (measured between virtual lines extending along the longitudinal extension directions of the first port and the second port) lies in a range of from 10° to 50°, in particular of from 15° to 45°, in particular of from 20° to 40°, in particular of from 10° to 30°. An angle in such a range allows a separation between the inner shaft and a part of the dividing element extending in a guiding direction in which a working catheter can be introduced into the guiding catheter through the first port, while avoiding an undesired bending of the inner shaft, in particular when moving it relatively to the outer shaft. An angle up to 30°, preferably up to 20°, is particularly appropriate to avoid buckling/kinking of the inner shaft.

In an embodiment, the dividing device is designed as Luer connector.

In an aspect, the present invention relates to a guiding catheter arrangement including a guiding catheter according to the preceding explanations and a working catheter that is introduced through the first port of the guiding catheter into the guiding catheter and extends through the slit into the second guiding lumen. Such a guiding catheter arrangement takes advantage of the specific design of the guiding catheter and enables a safe and reliable guidance of the working catheter through the second guiding lumen, wherein the length over which the working catheter is guided can be adjusted by advancing the inner shaft relatively to the outer shaft of the guiding catheter.

In an embodiment, the working catheter includes a stent and/or a balloon to be delivered by the working catheter to a site of action. As outlined above, such stent and/or balloon can be particularly easy, safe, and damaging-free delivered by the present guiding catheter arrangement.

In an aspect, the present invention relates to a method for extending an effective length of a guiding catheter. In an embodiment, methods for treatment of the human or animal body by surgery of therapy and diagnostic methods practiced on the human or animal body are explicitly excluded from the scope of protection. It should be noted that the method for extending an effective length of the guiding catheter can also be used outside the medical context in pure technical applications. In an alternative embodiment, however, the method for extending an effective length of the guiding catheter also includes medical methods for introducing a working catheter into a human or animal body.

The method for extending an effective length of the guiding catheter includes the steps explained in the following.

First, a guiding catheter according to the above given explanations is provided.

Afterwards, the outer shaft of the guiding catheter is advanced towards an intended site of action. Typically, there remains a gap of at least several centimeters between a distal terminus of the outer shaft and the intended site of action. Therefore, an extension of the effective guiding length of the guiding catheter is desired.

The outer shaft is then kept essentially immovable. In addition, the inner shaft is advanced relative to the outer shaft closer to the intended site of action. Consequently, a distal terminus of the inner shaft comes closer to the intended site of action than a distal terminus of the outer shaft.

In a further method step, a working catheter is advanced through the first port and through the slit into the second guiding lumen. This working catheter is then further guided within the second guiding lumen along a longitudinal extension direction of the inner shaft and finally through the distal terminus out of the second guiding lumen to the intended site of action.

For carrying out this method, it is not necessary to perform the individually explained method steps in the presently described sequence. Rather, any other logically possible sequence may also be applied.

In an embodiment, the relative position of the inner shaft with respect to the outer shaft is fixed by a fixing mechanism prior to advancing the working catheter through the second guiding lumen. This facilitates guiding the working catheter through the slit into the second guiding lumen and reduces the risk of any undesired relative movement of the inner shaft with respect to the working catheter. Such relative movement might be connected with a force exerted by the inner shaft and acting upon the working catheter. Such force is typically not desired since it may make an advancement of the working catheter more difficult and may result in a dislocation of the working catheter.

All embodiments of the guiding catheter can be combined in any desired way and can be transferred either individually or in any arbitrary combination to the guiding catheter arrangement and to the method. Likewise, all embodiments of the guiding catheter arrangement can be combined in any desired way and can be transferred either individually or in any arbitrary combination to the guiding catheter and to the method. Finally, all embodiments of the method can be combined in any desired way and can be transferred either individually or in any arbitrary combination to the guiding catheter and to the guiding catheter arrangement.

As mentioned above the guiding catheter can be used as guide extension catheter. Thus, a guide extension catheter is described including

an outer shaft defining a first guiding lumen in an interior of the outer shaft;
an inner shaft movably arranged at least partially in the first guiding lumen, wherein the inner shaft defines a second guiding lumen in an interior of the inner shaft and a dividing device arranged in a proximal end region of the guiding catheter, the dividing device having a first port and a second port angularly arranged to each other, wherein both the first port and the second port provide access to the first guiding lumen; wherein
the inner shaft extends through the second port into the first guiding lumen, wherein the inner shaft includes at least section-wise a slit along a longitudinal extension direction of the inner shaft, wherein the slit provides access to the second guiding lumen and enables a working catheter to enter the second guiding lumen from the first guiding lumen, wherein the inner shaft has a length exceeding a length of the outer shaft.

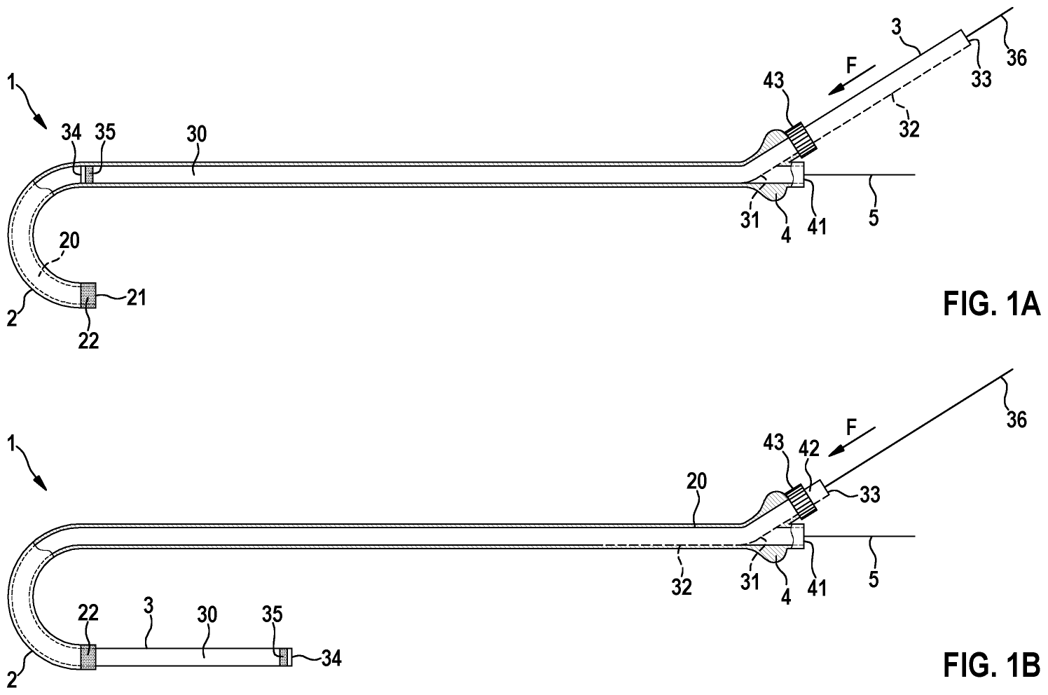

FIG. 1A shows a guiding catheter 1 having an outer shaft 2 and an inner shaft 3. An interior of the outer shaft 2 represents a first guiding lumen 20, whereas an interior of the inner shaft 3 represents a second guiding lumen 30. The inner shaft 3 is fed through a Luer connector 4 into the first guiding lumen 20. The Luer connector 4 serves as dividing device. It includes a first port 41 and a second port 42. The inner shaft 3 is fed through the second port 42 into the first guiding lumen 20 by axially advancing the inner shaft 3 along a feeding direction F. The feeding direction F corresponds to a longitudinal extension direction of the inner shaft 3.

A working catheter 5 is guided through the first port 41 into the Luer connector 4. This working catheter 5 is then guided through an opening 31 formed in the region of a slit 32 of the inner shaft 3 into the second guiding lumen 30. The slit 32 is positioned at an underside of the inner shaft 3 and extends along the feeding direction F in a proximal end region of the inner shaft 3 from a proximal terminus 33 of the inner shaft 3 up to a region where the inner shaft 3 is in a transition zone between the Luer connector 4 and the first guiding lumen 20 of the outer shaft 2.

In the operational state of the guiding catheter 1 shown in FIG. 1A, a distal terminus 34 of the inner shaft 3 is proximally arranged from a distal terminus 21 of the outer shaft 2. Therefore, the effective length of the guiding catheter 1 is defined by the length of the outer shaft 2.

In a very distal end region of the outer shaft 2 (directly proximally to the distal terminus 21 of the outer shaft 2), a first X-ray marker 22 is positioned. This first X-ray marker 22 facilitates a location of the guiding catheter 1 under X-ray control. Likewise, in a very distal end region of the inner shaft 3 (directly proximally to the distal terminus 34 of the inner shaft 3), a second X-ray marker 35 is arranged.

For stabilizing the inner shaft 3, an auxiliary wire 36 is at least partially fed through the second guiding lumen 30. The auxiliary wire 36 and the working catheter 5 are at least section-wise both positioned within the second guiding lumen 30.

If it turns out that the effective catheter length of the guiding catheter 1 is not sufficient to closely reach the site of action, i.e., the site at which a manipulation with the working catheter 5 is to be performed, the inner shaft 3 is advanced in the feeding direction F into the first guiding lumen 20. This situation is displayed in FIG. 1B. In this and in all following Figures, similar elements will be denoted with the same numeral reference.

Since the slit 32 is advanced together with the inner shaft 3, the opening 31 in the region of the Luer connector 4 remains open so that the working catheter 5 can always enter into the second guiding lumen 30 irrespective of the concrete relative position between the inner shaft 3 and the outer shaft 2.

The inner shaft 3 can be advanced into the first guiding lumen 20 by a length corresponding to the length of that portion of the inner shaft 3 that is proximally arranged of the second port 42 of the Luer connector 4. Of course, it is also possible to advance the inner shaft 3 into the first guiding lumen 20 only to a smaller extent than the full length being proximally to the second port 42.

In the situation displayed in FIG. 1B, basically the whole section of the inner shaft 3 that is proximally arranged to the second port 42 in the operational state of the guiding catheter 1 shown in FIG. 1A is pushed into the first guiding lumen 20. As a result, the distal terminus 34 of the inner shaft 3 protrudes from the distal terminus 21 of the outer shaft 2. Consequently, the guiding catheter 1 has a longer effective length than in case of the operational state shown in FIG. 1A. To be more precise, the effective length of the guiding catheter is extended by a distance that corresponds to the length of that portion of the inner shaft 3 that was arranged proximally to the second port 42 in the operational state shown in FIG. 1A.

When advancing the working catheter 5 through the second lumen 30, the working catheter 5 will exit the guiding catheter 1 through the distal terminus 21 of the outer shaft 2 in the operational state depicted in FIG. 1A, but only through the distal terminus 34 of the inner shaft 3 in the operational state depicted in FIG. 1B. Thus, it is possible to provide a safe guidance of the working catheter 5 by the guiding catheter 1 over a longer distance and thus to position the working catheter 5 very closely to the intended site of action.

To keep the inner shaft 3 in a stable and immovable position with respect to the outer shaft 3, the Luer connector 4 includes a twist lock 43. Upon opening the twist lock 43, it is possible to move the inner shaft 3 relatively to the outer shaft 2 as desired. Upon locking the twist lock 43, no such relative movement between the inner shaft 3 and the outer shaft 2 is any longer possible. Thus, once the inner shaft 3 has reached its intended position, it can be fixed with respect to the outer shaft 2 by locking the twist lock 43. Then, it is no longer necessary to manually fix the inner shaft 3. Rather, only one hand of the operator will be necessary to safely hold the guiding catheter 1 so that the operator's other hand can be used to advance the working catheter 5 to the intended site of action.

Since the inner shaft 3 is equipped with the second X-ray marker 35, the position of the inner shaft 3 with respect to a body tissue structure can be safely monitored during an introduction of the guiding catheter 1 into the body of a patient. The length by which the inner shaft 3 protrudes from the outer shaft 2 can be easily estimated by evaluating the relative positions of the first X-ray marker 22 and the second X-ray marker 35.

Once the working catheter 5 has been guided to the intended site of action and after having completed the manipulation to be performed with the working catheter 5, the working catheter 5 can be retracted through the guiding catheter 1 and be moved out of the body of the patient. During this procedure, the twist lock 43 can be released again to also retract the inner shaft 3 with respect to the outer shaft 2. Finally, the whole guiding catheter 1 can be removed out of the patient's body.

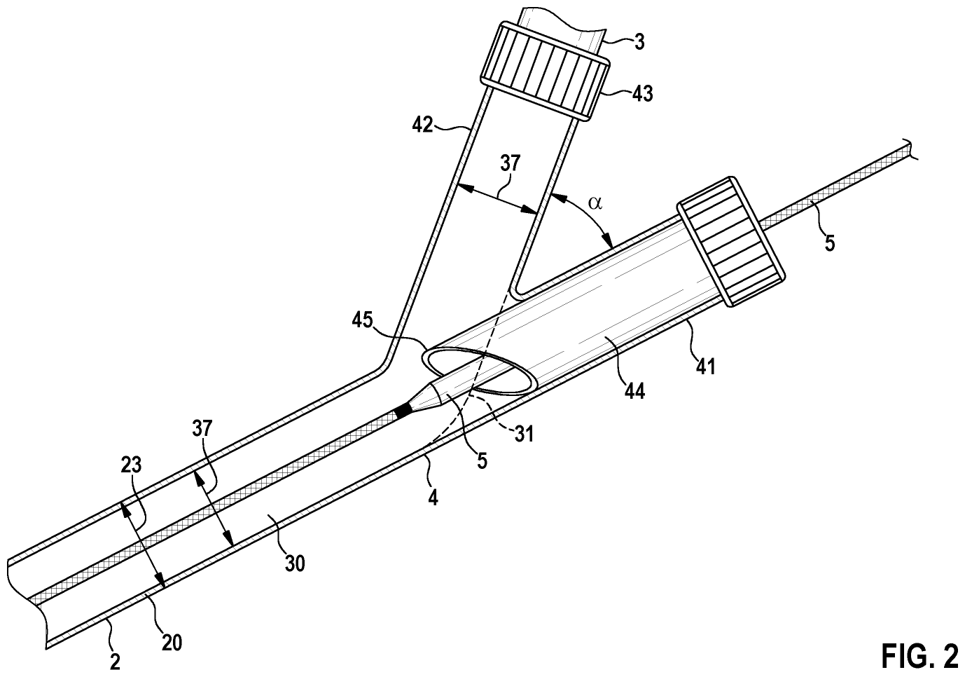

FIG. 2 shows a detailed view on the Luer connector 4 of the guiding catheter shown in FIGS. 1A and 1B. To facilitate the formation of the opening 31 in the region of the slit 32 of the inner shaft 3, through which opening 32 the working catheter 5 is guided into the second guiding lumen 30, the Luer connector 4 includes an access sleeve 44 that is inserted into the first port 41. The access sleeve 44 includes a tip 45 that protrudes into the second guiding lumen 30. The tip 45 of the access sleeve 44 separates the two wall sections of the inner shaft 3 lying on both sides of the slit 32. Thus, if the inner shaft 3 is advanced in the feeding direction F into the first guiding lumen 20, the tip 45 struts away the wall sections lying on both sides of the slit 32 and thus forms the opening 31 through which the working catheter 5 is guided into the second guiding lumen 30.

The access sleeve 44 also protects the working catheter 5 from a direct mechanical contact with the wall of the inner shaft 3 in the region of the slit since the wall of the inner shaft 3 slides over an outer surface of the access sleeve 44 when moving the inner shaft 3 along the feeding direction F.

The inner shaft 3 has a constant first diameter 37 of 5 French (1.67 mm). The outer shaft 2 has a constant second diameter 23 of 6 French (2 mm). Due to this diameter difference, the inner shaft 3 can be easily moved within the first guiding lumen 20, while avoiding any undesired clearance between an inner surface of the outer shaft 2 and an outer surface of the inner shaft 3.

An angle α between the first port 41 and the second port 42 amounts to approximately 40°. This angle α is chosen such that the inner shaft 3 can be well guided through the Luer connector 4 and the outer shaft 2 without being bent.

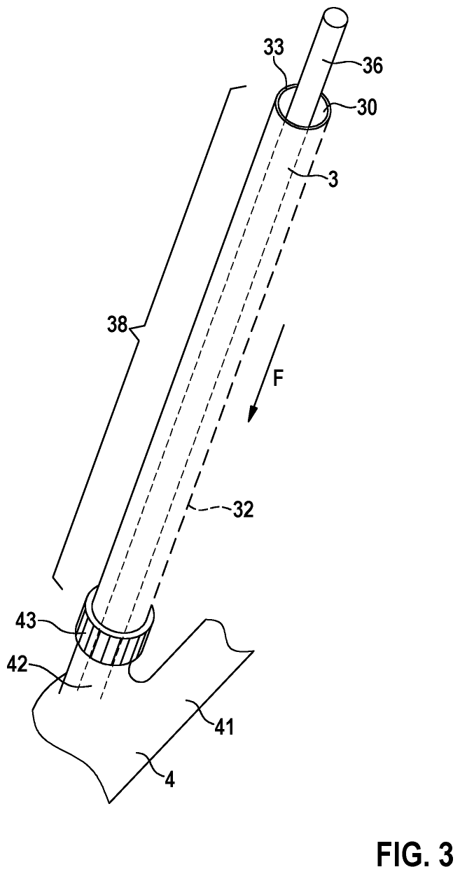

FIG. 3 shows a detail of the portion 38 of the inner shaft 3 that protrudes from the second port 42 to an outside of the guiding catheter 1. Here, the slit 32 on the underside of the inner shaft 3 can well be seen. The inner shaft 3 is positioned such that the slit 32 faces the access sleeve 44 (cf. FIG. 2) so that the opening 31 is formed by an interaction between the tip 45 of the access sleeve 44 and the slit 32 (also confer in this respect FIG. 2).

As outlined above, the inner shaft 3 is stabilized by the auxiliary wire 36 that is guided within the second guiding lumen 30. This auxiliary wire 36 helps in advancing the inner shaft 3 along the feeding direction F in a bend-free manner.

The twist lock 43 can be opened and closed as desired to allow, restrict or prohibit a relative movement between the inner shaft 3 and the outer shaft 2.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A guiding catheter comprising:
an outer shaft defining a first guiding lumen in an interior of the outer shaft;
an inner shaft movably arranged at least partially in the first guiding lumen, the inner shaft defining a second guiding lumen in an interior of the inner shaft; and
a dividing device arranged at a proximal end region of the guiding catheter, the dividing device comprising a first port and a second port angularly arranged with respect to each other, wherein both the first port and the second port provide access to the first guiding lumen; and wherein
the inner shaft extends through the second port into the first guiding lumen, wherein the inner shaft comprises at least a section-wise slit along a longitudinal extension direction (F) of the inner shaft, wherein the slit provides access to the second guiding lumen and enables a working catheter to enter the second guiding lumen from the first guiding lumen, wherein the inner shaft comprises a length exceeding a length of the outer shaft.

2. The guiding catheter according to claim 1, wherein the slit is present in a proximal end region of the inner shaft and comprises a length in a range of from 5 cm to 50 cm.

3. The guiding catheter according to claim 1, wherein the slit is present in a proximal end region of the inner shaft including a proximal terminus of the inner shaft.

4. The guiding catheter according to claim 1, wherein the dividing device comprises an access facilitation device providing access to the second guiding lumen through the slit.

5. The guiding catheter according to claim 4, wherein the access facilitation device comprises a tip penetrating the slit and increasing an area of an opening in a wall of the inner shaft formed by the slit.

6. The guiding catheter according to claim 4, wherein the access facilitation device is at least partially arranged inside the first port.

7. The guiding catheter according to claim 1, wherein the second port comprises a fixing device configured to fix the inner shaft relatively to the dividing device.

8. The guiding catheter according to claim 7, wherein the fixing device is configured to have an open position, in which a relative movement between the inner shaft and the outer shaft is possible; and a closed position, in which a relative movement between the inner shaft and the outer shaft is not possible.

9. The guiding catheter according to claim 1, wherein the guiding catheter comprises an auxiliary wire guided within the second lumen, wherein the auxiliary wire is configured to stabilize the inner shaft.

10. The guiding catheter according to claim 1, wherein the inner shaft comprises a length exceeding a length of the outer shaft by at least 10%.

11. The guiding catheter according to of claim 1, wherein the inner shaft comprises a same diameter over its entire length.

12. The guiding catheter according to claim 1, comprising one or both of an X-ray marker and a sensor at a distal end region of the inner shaft.

13. The guiding catheter according to claim 1, wherein an angle between the first port and the second port is in a range of from 10° to 50°.

14. The guiding catheter of claim 1, comprising a working catheter inserted through the first port into the guiding catheter and extending through the slit into the second guiding lumen.

15. (canceled)

16. A method for extending an effective length of a guiding catheter, comprising:
providing a guiding catheter according to claim 1;
advancing the outer shaft toward an intended site of action;
keeping the outer shaft essentially immovable;
advancing the inner shaft relative to the outer shaft further to the intended site of action and thus coming closer to the intended site of action with a distal terminus of the inner shaft than with a distal terminus of the outer shaft; and
advancing a working catheter through the first port and through the slit into the second guiding lumen and further through the distal terminus out of the second guiding lumen to the intended site of action.

17. The method according to claim 16, wherein a relative position of the inner shaft with respect to the outer shaft is fixed before advancing the working catheter through the second guiding lumen.

18. The guiding catheter of claim 13, wherein the angle is in a range from 10° to 30°.

19. The guiding catheter of claim 18, wherein the angle is in a range from 10° to 20°.
